(19) United States
(12) Patent Application Publication
(10) Pub. No.: US 2020/0098476 A1
(43) Pub. Date: Mar. 26, 2020

(54) DYNAMIC PROMPTING FOR DIAGNOSIS SUSPECTING

(71) Applicant: Clover Health, Jersey City, NJ (US)

(72) Inventors: Peter Vladimir Loscutoff, Berkeley, CA (US); Ian Blumenfield, Oakland, CA (US); Christopher James Lauinger, Golden, CO (US); Melanie Goetz, Oakland, CA (US)

(21) Appl. No.: 16/141,732

(22) Filed: Sep. 25, 2018

(51) Int. Cl. G16H 50/20; G06F 17/30; G16H 10/60

(52) U.S. Cl. CPC G16H 50/20; G16H 10/60; G06F 17/30876

(57) ABSTRACT

Systems and methods including analyzing profiles, generating questions relate to suspected diagnoses of a patient, and transmitting the questions to a device that displays the questions are disclosed. Responses to the questions are received which may be used in subsequent suspected diagnosis, generating updated questions, or confirming a suspected diagnosis.

### BACKGROUND

Doctors, nurses, or other medical professionals often examine patients to determine health related issues. Examination(s) may include in-person visits (e.g., hospital or in-home), over the phone, and/or virtually. During the examination, the medical professionals may ask the patient questions and/or perform tests. Determining the questions to ask and/or the tests to perform, for instance, may be important in properly diagnosing a patient and/or identifying future measures to take. Described herein are improvements in technology and solutions to technical problems that may be used, among other things, to increase the materiality of patient examinations.

### BRIEF DESCRIPTION OF THE DRAWINGS

The detailed description is set forth below with reference to the accompanying figures. In the figures, the left-most digit(s) of a reference number identifies the figure in which the reference number first appears. The use of the same reference numbers in different figures indicates similar or identical items. The systems depicted in the accompanying figures are not to scale and components within the figures may be depicted not to scale with each other.

FIG. 1 illustrates an example of dynamic prompting for diagnosis suspecting in an environment. The environment may include a medical professional and a patient, whereby the medical professional administers question(s) displayed by a device. In some instances, the device may receive the question(s) from remote computing resource(s).

FIG. 2 illustrates a block diagram of selected functional components of the computing resource(s) of FIG. 1.

FIG. 3 illustrates a block diagram of selected functional components of the device of FIG. 1.

FIG. 4 illustrates a flow diagram of an example process of the remote computing resource(s) of FIG. 1 generating question(s), receiving response(s), and generating updated question(s).

FIG. 5 illustrates a flow diagram of an example process of the device of FIG. 1 displaying question(s), receiving response(s), and transmitting the response(s).

FIG. 6 illustrates a signal diagram for transmitting question(s) and receiving response(s) to generate updated question(s).

FIG. 7 illustrates an example process of updating the device of FIG. 1 to display question(s).

FIG. 8 illustrates an example process of displaying suspected diagnoses and transmitting responses.

### DETAILED DESCRIPTION

Systems and methods of dynamic prompting for diagnosis suspecting are described herein. In diagnosing a patient with an illness, disease, condition, or sickness, medical professionals (e.g., doctor, nurse, physician's assistant, nurse practitioner, etc.) may ask the patient questions about his or her medical history such as their previous symptoms, diagnoses, doctor visits, and so forth. Charts detailing a patient's medical history may also be used in diagnosing, such as results from tests (e.g., blood tests, Electrocardiography (EKG), etc.). However, a patient's medical history, such as the charts, in some instances may fail to indicate diagnoses and/or correlate certain symptoms with a disease. For instance, a patient may experience symptoms associated with a disease (e.g., diabetes) but may have never been diagnosed and/or treated for the disease. Additionally, patients may improperly recall past symptoms and/or provide dishonest answers to questioning performed by medical professionals. As a result of the foregoing, medical professionals may ask unrelated, irrelevant, or inapplicable questions to patients and/or may improperly correlate certain symptoms with diagnoses, potentially leading to a misdiagnosis or a failure to diagnose.

In light of the above, the present application describes providing prompts or questions used by a medical professional when examining and/or interacting with a patient. The questions may be provided to a device operated by the medical professional, such as a tablet, computer, or phone, and the device may be configured to display the questions. The medical professional may then present, whether audibly and/or visually, the questions to the patient. Thereafter, feedback, answers, or responses to the questions may be entered on the device. In some instances, these responses may indicate whether the patient is suspected of having diagnoses. For instance, the question(s) may ask whether the patient is suspected of having diabetes, Alzheimer's, post-traumatic stress disorder (PTSD), epilepsy, and so forth. After examining the patient, the medical professional may provide response(s) to these question(s). In some instances, the answers may be entered by the patient. Alternatively, or additionally, the device may include a text-to-speech component that audibly outputs the questions and a speech-to-text component that converts a patient's response into text.

The questions displayed on the device may be received and/or generated from a remote computing resource(s) (e.g., cloud, server, etc.) and the questions may be tailored according to the patient's medical history, symptoms, and/or personal information. As an example, the remote computing resource(s) may include (e.g., store) user profiles corresponding to patients and/or one or more databases associated with medical records, news, diagnostics, statistics, and/or other medical information. The remote computing resource(s) may analyze the user profiles, such as a medical history of the patient, and/or the one or more databases to determine question(s) to ask a patient. In some instances, the question(s) may relate to one or more suspected diagnoses of the patient.

The remote computing resource(s) may employ machine learning algorithms or techniques to generate the question(s). In some instances, the machine leaning techniques may correlate a patient's medical history or historical trends with one or more suspected diagnoses of the patient, despite, in some instances, the patient's medical history (or other information) failing to indicate the suspected diagnoses. More specifically, while a patient's medical history may include symptoms associated with an illness, these symptoms, individually, may not be correlated to a suspected diagnosis. In this sense, the machine learning techniques function to aggregate and analyze trends in a patient's medical history as well as, in examples, trends in other patients' medical histories, to determine one or more suspected diagnoses. As such, the question(s) posed to the patient may be specific to his or her prior medical history and inquire about past and current symptoms and/or diagnoses in order to determine whether the patient is suspected of having a particular diagnosis. In some instances, the questions may relate to triaging the one or more suspected diagnoses of the patient, may inquire about previous medical symptoms to determine one or more suspected diagnoses, may relate to a determination of whether the patient is suspected of having a diagnosis, and/or may otherwise be used to identify health related concerns of the patient.

The device may display the questions for the medical professional to utilize when examining a patient. For instance, after analyzing the user profiles and/or the databases, the questions may relate to one or more potential suspected diagnoses of the patient (e.g., diabetes, heart disease, etc.). While the medical professional is examining the patient, the medical professional may make an assessment as to whether the patient has the one or more suspected diagnoses. That is, the medical professional may provide response(s) to the questions “Does the patient have diabetes” and/or “Does the patient have heart disease?” In making this assessment, the medical professional may ask questions, perform tests, and so forth before providing an indication as to the suspected diagnoses.

In some instances, the device may display the questions and upon receiving responses, the device may transmit data corresponding to the responses to the remote computing resource(s). The data may be analyzed by the remote computing resource(s) and utilized to generate updated questions and/or analyze trends for future diagnoses suspected in additional patients. For instance, the machine learning techniques may analyze the responses, the user profiles, and/or the database to determine updated question(s) to ask the patient. Additionally, if the question asks “Does the patient have diabetes” and the response is “No,” the machine learning techniques may correlate symptoms and/or a medical history of this particular user (which was initially suspected of having diabetes) when determining suspected diagnoses of future patients having a similar medical history as the particular user. That is, the techniques herein may learn the response and use these indications for future diagnoses suspecting. In some instances, after receiving the responses, the remote computing resource(s) may transmit the updated questions to the device and the medical professional may utilize the updated questions when interacting with the patient.

Given the communicative relationship between device and the remote computing resource(s), the questions posed to the patient may be updated in real time and according to the responses provided by the patients. That is, the device may transmit the responses and may receive, substantially contemporaneously with transmitting the responses, updated questions. In other words, the remote computing resource(s) may continuously generate and transmit, substantially contemporaneously with receiving the responses, updated questions. In doing so, using the machine learning techniques described herein, the questions posed to the user may be refined according to the responses, thereby assisting in determining or helping to refine one or more suspected diagnoses of the patient. In some instances, the device may transmit one or more responses to one or more respective questions individually, or the responses may be transmitted as a batch. Questions transmitted by the remote computing resource(s) may be carried out in a similar fashion.

To illustrate the transmittal and updating of questions, at a first instance, the remote computing resource(s) may provide initial questions to the device. Noted above, these initial questions may be generated specific to a patient through analyzing the patient's medical history and/or the questions may be generated based on analysis of one or more machine learning algorithms utilizing data from other patients. For instance, after analyzing the patient's medical record and/or the data from other patients, it may be determined that diabetes and epilepsy are suspected diagnoses of the patient. Using this determination, the questions provided to the medical professional may ask the medical professional to either confirm or deny the suspected diagnoses. That is, after examining the patient, the medical professional makes an assessment as to whether the patient has diabetes or epilepsy, for instance. Additionally, questions related to determining whether the patient has diabetes and/or epilepsy (or another disease) may be generated and transmitted to the device. For instance, the device may display one or more of the initial questions, such as “Have you been taking any medication?” and/or “When is the last time you experienced a seizure?” The device may receive responses which may include “Yes, I've been taking insulin” and/or “I haven't experienced a seizure in three years.” Upon transmitting these responses to the remote computing resource(s), the remote computing resource(s) may analyze the responses and generate updated questions based on the response(s). For instance, using the responses, the remote computing resource(s) may determine to further inquire about diabetes, given the patient's answer and taking insulin, but not epilepsy given the patient's indication of the last occurrence of a seizure. Moreover, the remote computing resource(s) may store indications of whether the patient has the suspected diagnoses (as determined from the medical professional). Noted above, these indications may be used by the machine learning techniques when performing diagnostics on future patients.

In some instances, after analyzing the responses, the remote computing resource(s) may generate updated questions relating to diabetes in order to further determine whether diabetes is a suspected diagnosis of the patient. That is, rather than posing additional questions related to epilepsy, for instance, the updated questions generated by the remote computing resource(s) may relate to determining whether the patient is suspected to be diabetic. Therefore, in receiving the updated questions at a second instance, instead of displaying subsequent questions of the initial questions, the device may replace these questions with the updated questions. In other words, some or all of the initial questions may not be responded to, but instead, the questions may be replaced with potentially more material questions related to the suspected diagnoses. More generally, in receiving a first response to a first question, the device may receive (from the remote computing resource(s)) an updated second question (based on the first response), before displaying an initial second question. As such, responses may be provided to the updated questions, which may afterward be transmitted to the remote computing resource(s) and used to generate additional updated questions.

With the above process, the device and the remote computing resource(s) may be in communication to receive and generate, respectively, iterative updated questions. After a sufficient amount of questions are generated and after a sufficient amount of responses are received, the remote computing resource(s) (or the device), may indicate one or more suspected diagnoses of the patient. In some instances, the one or more suspected diagnoses may be determined after a threshold amount of questions are posed, after a threshold amount of responses are received, after a confidence or probability level of the one or more suspected diagnoses exceeds a threshold, and/or any combination thereof. For instance, through iteratively posing updated questions, the remote computing resource(s) may indicate probabilities (or confidences) that the patient has one or more suspected diagnoses. After this probability exceeds a threshold, the remote computing resource(s) may generate and/or store an indication in the user profile of the patient. As will be understood, the iterative process of updating questions further refines and identifies, through the received responses, one or more suspecting diagnoses of the patient.

Compared to conventional techniques, which include predefined or static questions, or fail to correlate certain symptoms with suspected diagnoses, the process described herein provides for the real-time generation and transmittal of updated questions. Such real-time updating is crucial given the time-sensitive interaction with patients and the time-sensitive nature of diagnosing patients. In other words, as medical professionals often have limited time with patients, the questions generated must be updated substantially close in time with the received responses. By way of comparison, if the questions are updated after the medical professional is no longer interacting with the patient, or if the medical professional forgets to ask certain questions, further examinations or appointments may be required. Such a process, however, is inefficient and may potentially harm the patient. Instead the system and methods described herein allow for the time-sensitive generation and transmittal of updated questions before presenting unrelated, irrelevant, or immaterial questions of an initial set of questions (based on the patient's responses to the questions). Moreover, through analyzing the user profiles, the databases, and the responses, the instant application allows for more flexibility in generating questions for patients, allowing the questions to be updated on-the-fly. With this, as the remote computing resource(s) continuously receive responses to updated questions, and through the analysis performed by the machine learning techniques, for instance, the updated questions are able to integrate a patient's responses in determining updated questions. Still, the processes herein utilize the response(s) provided by patients when determining one or more suspected diagnoses of future patients. This iterative refinement of using the responses to update suspected diagnoses of patients is an efficient use of computing resources as symptoms of patients are correlated to one another and in future instances the one or more suspected diagnoses may be more accurately determined. Noted above, this process would not be possible in conventional techniques given the static nature of the questions, the inability to update questions in real-time, and the time-sensitive nature of determining correlations between patients when determining suspected diagnoses. That is, the analysis performed by the machine learning technique in generating trends, historical models, comparing user profiles, comparing database(s) would not otherwise be possible in conventional methods given the vast amount of information that is required to be analyzed in such a time-sensitive manner.

The present disclosure provides an overall understanding of the principles of the structure, function, manufacture, and use of the systems and methods disclosed herein. One or more examples of the present disclosure are illustrated in the accompanying drawings. Those of ordinary skill in the art will understand that the systems and methods specifically described herein and illustrated in the accompanying drawings are non-limiting embodiments. The features illustrated and/or described in connection with one embodiment may be combined with the features of other embodiments, including as between systems and methods. Such modifications and variations are intended to be included within the scope of the appended claims. Additional details are described below with reference to several example embodiments.

### Illustrative Environment

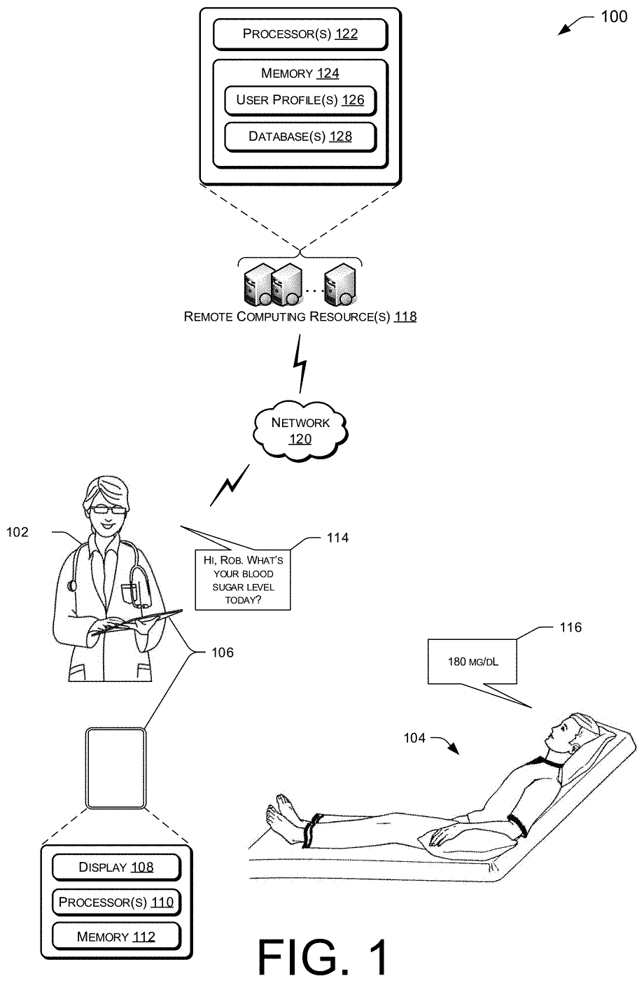

FIG. 1 shows an illustrative environment 100 which may include a provider 102 and a patient 104. In some instances, the environment 100 may be located at a medical facility (e.g., hospital, clinic, etc.) or at a residence of the patient 104. The environment 100 may also include a device 106 with which the provider 102, or in some instances, the patient 104 may interact. In the illustrative implementation, the provider 102 is holding the device 106. In other implementations, the patient 104 may hold the device 106. Further, more than one device 106 may be included within the environment 100. For instance, the provider 102 may have a device 106 while the patient 104 may have a separate device 106. In such instances, the devices may be configured to communicate with one another.

The device 106 may include a display 108 to display content. In some instance, the display 108 may include a touchscreen capable of receiving input from the provider 102 (or the patient 104). For instance, the display 108 may include a graphical user interface (GUI) that receives input from the provider 102. The display 108 may also include a virtual keyboard, buttons, input fields, and so forth, to permit the provider 102 to interact with the device 106.

The device 106 includes processor(s) 110 and memory 112. Discussed in detail herein, the processor(s) 110 may configure the device 106 to present questions on the display 108. Therein, the provider 102 may ask the questions to the patient 104, may perform examination(s) or diagnostics related to the questions, and/or enter a response on the device 106. For instance, FIG. 1 illustrates the provider 102 asking a question 114: “Hi, Rob. What's your blood sugar level today?” The patient 104 thereafter provides a response 116: “180 mg/dL.” The provider 102 enters or records the response 116 into an input field on the display 108. Thereafter, in some instances, the device 106 may present additional questions, which the provider 102 subsequently relates to the patient 104. Additionally, or alternatively, the question may relate to or ask, “Does the patient have diabetes?” Therein, the provider 102 may use this prompt to determine whether the patient 104 has diabetes. For instance, the provider may ask questions (whether prompted by the device 106 or from the provider's 102 training) to make an assessment whether the patient 104 is suspected of having diabetes. This response may therein be used for future diagnosis suspecting and/or to present additional questions to the patient 104.

The device 106 may be communicatively coupled to one or more remote computing resource(s) 118 to receive the questions, such as the question 114. Additionally, the device 106 may transmit responses, such as the response 116 to the remote computing resource(s) 118. The remote computing resource(s) 118 may be remote from the environment 100 and the device 106. For instance, the device 106 may communicatively couple to the remote computing resource(s) 118 over a network 120. In some instances, the device 106 may communicatively couple to the network 120 via wired technologies (e.g., wires, USB, fiber optic cable, etc.), wireless technologies (e.g., RF, cellular, satellite, Bluetooth, etc.), or other connection technologies. The network 120 is representative of any type of communication network, including data and/or voice network, and may be implemented using wired infrastructure (e.g., cable, CATS, fiber optic cable, etc.), a wireless infrastructure (e.g., RF, cellular, microwave, satellite, Bluetooth, etc.), and/or other connection technologies.

The remote computing resource(s) 118 may be implemented as one or more servers and may, in some instances, form a portion of a network-accessible computing platform implemented as a computing infrastructure of processors, storage, software, data access, and so forth that is maintained and accessible via a network such as the Internet. The remote computing resource(s) 118 do not require end-user knowledge of the physical location and configuration of the system that delivers the services. Common expressions associated with these remote computing resource(s) 118 may include “on-demand computing,” “software as a service (SaaS),” “platform computing,” “network-accessible platform,” “cloud services,” “data centers,” and so forth.

The remote computing resource(s) 118 include a processor(s) 122 and memory 124, which may store or otherwise have access to one or more user profile(s) 126 and/or one or more database(s) 128. Discussed in detail herein, the remote computing resource(s) 118 may generate and transmit the questions to the device 106 and in generating the questions, the remote computing resource(s) 118 may utilize the user profile(s) 126 and/or the database(s) 128. The remote computing resource(s) 118 may also receive the responses, and utilize the responses to generate additional or updated questions, as well as using the response for future diagnosis suspecting. The updated questions may be transmitted to the device 106. Through this interaction, questions may be posed to the patient 104 and responses may be received from the patient 106. In turn, the responses may be used by the remote computing resource(s) 118 to identify one or more suspected diagnoses of the patient and/or a probability thereof.

As used herein, a processor, such as processor(s) 110 and/or 122, may include multiple processors and/or a processor having multiple cores. Further, the processors may comprise one or more cores of different types. For example, the processors may include application processor units, graphic processing units, and so forth. In one implementation, the processor may comprise a microcontroller and/or a microprocessor. The processor(s) 110 and/or 122 may include a graphics processing unit (GPU), a microprocessor, a digital signal processor or other processing units or components known in the art. Alternatively, or in addition, the functionally described herein can be performed, at least in part, by one or more hardware logic components. For example, and without limitation, illustrative types of hardware logic components that may be used include field-programmable gate arrays (FPGAs), application-specific integrated circuits (ASICs), application-specific standard products (ASSPs), system-on-a-chip systems (SOCs), complex programmable logic devices (CPLDs), etc. Additionally, each of the processor(s) 110 and/or 122 may possess its own local memory, which also may store program components, program data, and/or one or more operating systems.

The memory 112 and/or 124 may include volatile and nonvolatile memory, removable and non-removable media implemented in any method or technology for storage of information, such as computer-readable instructions, data structures, program component, or other data. Such memory 112 and/or 124 may include, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium which can be used to store the desired information and which can be accessed by a computing device. The memory 112 and/or 124 may be implemented as computer-readable storage media (“CRSM”), which may be any available physical media accessible by the processor(s) 110 and/or 122 to execute instructions stored on the memory 112 and/or 124. In one basic implementation, CRSM may include random access memory (“RAM”) and Flash memory. In other implementations, CRSM may include, but is not limited to, read-only memory (“ROM”), electrically erasable programmable read-only memory (“EEPROM”), or any other tangible medium which can be used to store the desired information and which can be accessed by the processor(s).

### Illustrative Remote Computing Resources

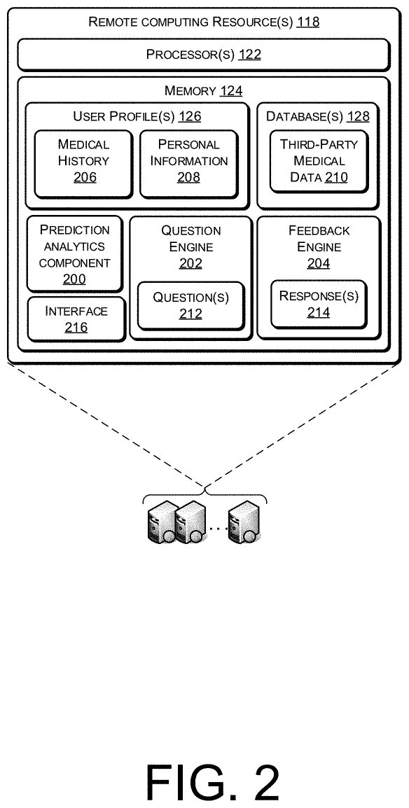

FIG. 2 shows selected functional components of the remote computing resource(s) 118. The remote computing resource(s) 118 includes the processor(s) 122 and the memory 124. As illustrated, the memory 124 of the remote computing resource(s) 118 stores or otherwise has access to user profile(s) 126, the database(s) 128, a prediction analytics component 200, a question engine 202, and a feedback engine 204. The user profile(s) 126 may correspond to a respective user (e.g., patients). Each user profile 126 may include a user's medical history 206 and personal information 208. In some instances, the medical history 206 may include a medical history of the user, such as diagnoses (e.g., disease, illness, etc.), treatments (e.g., medications, surgeries, therapy, etc.), family medical history (e.g., diabetes, Alzheimer's, etc.), measurements (e.g., weight, height, etc.), symptoms (e.g., sore throat, back pain, loss of sleep, etc.), and so forth. The personal information 208 may include names (e.g., social security number (SSN)), identifiers, residence, work history, acquaintances, heritage, age, and so forth. The medical history 206 and/or the personal information 208 may be received using record locators and/or searching databases.

The database(s) 128 may include information or third-party medical data 210 obtained from third-party sources. The third-party sources may include a source (or service) that collects, stores, generates, filters, and/or provides medical news. In some instances, the third-party sources that provide the third-party medical data 210 may include news agencies (e.g., ABC, NBC, CBS, FOX, BBC, CNN, etc.), governmental agencies or services (e.g., U.S. Department of Health and Human Services (HHS), Centers for Disease Control and Prevention (CDC), National Institute of Health (NIH), etc.), medical new websites or sources (e.g., webmd.com, etc.), other medical sources (e.g., American Red Cross, Universities, Hospitals, etc.). The third-party medical data 210 may also include data obtained from other online resources that search for content, such as medical information. For instance, the online resources may include, but are not limited to, search engines (e.g., GOOGLE®), social media sites (e.g., FACEBOOK®, INSTRAGRAM®, etc.), databases, and/or other online resources. The remote computing resource(s) 118 may be in communication with the third-party sources to obtain, retrieve, and/or receive the third-party medical data 210 representing medical situations, medical conditions, and/or medical news.

As noted above, the remote computing resource(s) 118 may analyze the user profile(s) 126 and/or the database(s) 128 to generate questions and/or determine one or more suspected diagnoses of a patient. For instance, the question engine 202 may generate question(s) 212 after the prediction analytics component 200 analyzes the user profile(s) 126 and/or the database(s) 128 to determine appropriate questions 212 to ask a user. That is, the prediction analytics component 200 may analyze the user profile(s) 126 and/or the database(s) 128 when determining the question(s) 212 and/or suspected diagnoses of the patient. The prediction analytics component 200 may be configured to determine one or more topics, issues, and/or question(s) 212 to be generated by the question engine 202. Stated alternatively, the prediction analytics component 200 functions to determine question(s) 212 that should be asked of the patient in determining one or more suspected health concerns (or diagnoses) of the patient or whether the patient is suspected of having particular diagnoses. For instance, based on analyzing the user profile(s) 126 and/or the database(s) 128, the prediction analytics component 200 may identify suspected diagnoses of the patient. In some instances, the analysis may involve comparing symptoms stored in the user profile(s) 126 to the database(s) 128 (or other user profile(s) 126) to determine correlations between the patient's symptoms and one or more suspected diagnoses. That is, continuing with the above example, based on the analysis, the prediction analytics component 200 may determine that symptoms of a patient correlate closely with one or more diagnoses.

Additionally, or alternatively, the prediction analytics component 200 may determine the suspected diagnoses despite the user profile(s) 126 failing to indicate such diagnoses. For instance, the user profile 126 of a patient may indicate two distinct symptoms, such as a first symptom (e.g., high blood sugar levels) and a second symptom (e.g., skin infections). These symptoms may be analyzed by the prediction analytics component 200 to determine that the patient is suspected of having diabetes. However, taken individually, these symptoms may fail to indicate that diabetes is a suspected diagnosis. In other words, individually, the first symptom and the second symptom may not indicate that the patient has diabetes and/or the first symptom and the second symptom may not indicate the probability of the suspected diagnosis over a threshold. Using the prediction analytics component 200, the symptoms of a patient may be aggregated and correlated to symptoms associated with a suspected diagnosis (e.g., diabetes). That is, when looked at collectively, the prediction analytics component 200 may determine that the first symptom and the second symptom may be indicative of diabetes. Using this determination, the prediction analytics component 200 may identify further information that is needed before determining a probability of the diagnoses. To obtain this information, the question engine 202 may generate question(s) 212.

The question(s) 212 generated relate to and/or utilize the outcomes of the prediction analytics component 200. Predictive analytic techniques may include, for example, predictive questioning, machine learning, and/or data mining. Generally, predictive questioning may utilize statistics to predict outcomes and/or question(s) to propose in future. Machine learning, while also utilizing statistical techniques, provides the ability to improve outcome prediction performance without being explicitly programmed to do so. Any number of machine learning techniques may be employed to generate and/or modify the question(s) 212 describes herein. Those techniques may include, for example, decision tree learning, association rule learning, artificial neural networks (including, in examples, deep learning), inductive logic programming, support vector machines, clustering, Bayesian networks, reinforcement learning, representation learning, similarity and metric learning, sparse dictionary learning, and/or rules-based machine learning.

Information from stored and/or accessible data (e.g., the medical history 206, the personal information 208, and/or the third-party medical data 210, etc.) may be extracted from the user profile(s) 126 and/or the database(s) 128 and utilized by the prediction analytics component 200 to predict trends and behavior patterns. The predictive analytic techniques may be utilized to determine associations and/or relationships between explanatory variables and predicted variables from past occurrences and utilizing these variables to predict the unknown outcome. The predictive analytic techniques may include defining the outcome and data sets used to predict the outcome. In defining the outcome, the prediction analytics component 200 may identify or determine question(s) 212 to further determine associations and/or relationships between explanatory variables and predicted variables. That is, data provided by the response(s) 214 may be collected and/or accessed to be used for analysis. Data analysis may include using one or more models, including for example one or more algorithms, to inspect the data with the goal of identifying useful information and arriving at one or more determinations that assist in predicting the outcome of interest. One or more validation operations may be performed, such as using statistical analysis techniques, to validate accuracy of the models. Thereafter predictive modelling may be performed to generate accurate predictive models for future events. By so doing, the prediction analytics component 200 may utilize data from the user profile(s) 126 and/or the database(s) 128, as well as features from other systems as described herein, to predict or otherwise determine a probability of one or more suspected diagnoses. Certain variables (e.g., symptoms) of the patient may be weighed more heavily than other symptoms in determining the outcome. Outcome prediction may be deterministic such that the outcome is determined to occur or not occur. Additionally, or alternatively, the outcome prediction may be probabilistic of whether the outcome is determined to occur to a certain probability and/or confidence.

Importantly, in utilizing outcomes of the prediction analytics component 200, the processor(s) 122 may configure the question engine 202 to generate the question(s) 212. In some instances, the question(s) 212 may include multiple choice questions (e.g., “Is you pain level (A) 1-2, (B) 3-4, (C) 5-6, (D) 7-8, or (E) 9-10?”), survey questions (e.g., “Have you or any of your family members had cancer? Yes ( ) No ( )” or “Does the patient have anxiety?”), true/false questions (e.g., “Have you missed a prescription?”), inquiry questions (e.g., “What is your blood sugar level after eating?” or “Explain the symptoms you have experienced over the past month?”), and so forth. In some instances, the question(s) may be phrased from the perspective of the provider 102 asking the patient 104 (e.g., “Ask the patient questions to determine whether they have depression” or may be phrased directly to the patient 104 (e.g., “Have you experienced mood swings?”). After generating the question(s) 212, the processor(s) 122 may transmit data corresponding to the question(s) 212 to the device 106. Discussed herein, upon receipt of the question(s) 212, the device 106 is configured to display the question(s) 212, such as on the display 108.

The question(s) 212 may be transmitted to the device 106 in response to a pull request from the device 106. Additionally, or alternatively, the question(s) 212 may be pushed to the device 106 after generating the question(s) 212. The remote computing resource(s) 118 may transmit the question(s) 212 with a command that causes the device 106 to display the question(s) 212. To communicate with the device 106, the third-party sources providing the third-party data 210, or other entities, the remote computing resource(s) 118 include an interface 216.

The remote computing resource(s) 118 are configured to receive, from the device 106, answers, prompts, messages, feedback, or response(s) 214 (e.g., words, phrase, sentences, etc.) to the question(s) 212. In some instances, the response(s) 214 may be received by the feedback engine 204 and/or the processor(s) 122 may forward the response(s) 214 to the feedback engine 204. Upon receiving the response(s) 214, the feedback engine 204 may be configured to the analyze the response(s) 214 to determine words, phrases, and expressions contained therein. For instance, if the question 212 asks “What is your blood sugar level after eating?” and the response 214 includes “170 mg/dL,” the feedback engine 204 may extract this value and provide the value to the prediction analytics component 200. As another example, if the question 212 asks “Does the patient have diabetes” and the response 214 includes the answer “No,” the feedback engine 204 may extract this value. Therein, the prediction analytics component 200 may utilize the response 214 to predict outcomes, correlations, or other relationships.

For instance, the prediction analytics component 200 may utilize the user profile(s) 126, the database(s) 128, and/or the response(s) 214 to determine that “170” is a normal and/or healthy blood sugar level after eating. In some instances, this determination may result from comparing the value with the user profile(s) 126 and/or the database(s) 128. For instance, the prediction analytics component 200 may compare “170 mg/dL” to determine that other patients having this blood sugar level were not diagnosed with diabetes, thereby utilizing correlations between other patients and their symptoms. However, it is to be understood that the prediction analytics component 200 may utilize more than a single response in making the determination. In turn, through the comparison, the prediction analytics components 200 may determine that the patient is not suspected of having diabetes. With this indication, the prediction analytics component 200 may determine other question(s) 212 that should be posed to the patient. For instance, while the patient might not be suspected of having diabetes, the prediction analytics component 200 may determine the patient should be asked about their blood pressure (e.g., through analyzing a family history of the patient stored in the user profile 126). In this sense, the prediction analytics component 200 may adapt the question(s) 212 through analyzing the response(s) 214 and determining a change or shift in focus of suspected diagnoses of the patient. More particularly, using the response(s) 214, the prediction analytics component 200 may determine an increase or decrease in a probability of the one or more suspected diagnoses. Such results are then used by the question engine 202 to generate the question(s) 212.

If, however, the response 214 to “What is your blood sugar level after eating?” is “240,” the prediction analytics component 200 may determine that this blood sugar level is concerning and suspect that the patient might be diabetic. As a result, the prediction analytics component 200 may determine that further question(s) 212 should be asked of the patient to determine whether the patient is suspected of being diabetic and/or a probability thereof. That is, before suspecting the patient may be diabetic, the prediction analytics components 200 may require further information, such as whether the patient is experiencing blurred vision, fatigue, and/or weight loss (i.e., other common symptoms of diabetic patients). The question engine 202 may utilize these determinations in generating updated question(s) 212, such as, for instance, “Have you recently experienced blurred vision?”

Noted above, certain symptoms may be weighed by the prediction analytics component 200 in determining the question(s) 212 and/or the suspected diagnoses. For instance, a blood sugar level of 240 mg/dL may be weighed more heavily in determining a probability of the patient being diabetic, as compared to whether the patient is experienced blurred vision.

The response(s) 214 may be used by prediction analytics component 200 to assist in identifying one or more suspected diagnoses more accurately in the future. For instance, based on the response(s) 214, the prediction analytics component 200 may correlate, map, and/or identify certain symptoms or characteristics with one or more suspecting diagnoses. As an example, if the prediction analytics component 200 consistently, or over a threshold amount of times, receives response(s) 214 that males between the ages of 30-35 having a blood pressure of 120/80 have not been diagnosed with heart disease, the prediction analytics component 200 may utilize this indication to determine that future patients having a similar blood pressure are not likely susceptible to having heart disease. As a result, when future question(s) 212 inquire about heart disease, the prediction analytics component 200 may apply this deduction in determining the probability of a patient has heart disease. Further, if certain symptoms of patients indicate that heart disease is a suspected diagnosis (such that the question(s) 212 generated relate to heart disease), but in assessing the patient 104 the provider 102 determines that a particular patient does not have heart disease, the prediction analytics component 200 may use these response(s) 214 (e.g., “No”) to train models for future diagnoses suspecting of additional patients. In other words, with the response(s) 214, symptoms that the prediction analytics component 200 used to determine that the patient was suspected of having heart disease may be analyzed for use in determining whether future patients having similar symptoms are not suspected of having a diagnosis (e.g., heart disease).

The prediction analytics component 200 may also reference other diagnoses and/or systems stored in other user profile(s) 126. In this sense, the prediction analytics component 200 may compare symptoms of a respective patient with symptoms experienced by other patients in determining suspected diagnoses and mapping the user profile(s) 126 together and analyzing trends. For instance, other patients may have experienced similar symptoms as the patient and the prediction analytics component 200 may use these indications to determine suspected diagnoses of the patient. In some instances, the amount of influence this factor has may decay over time. For instance, if two patients are experiencing similar symptoms and one was diagnosed with diabetes within a year, then the prediction analytics component 200 may weight this interaction more greatly than if the diagnosis was several years prior.

In some instances, after determining one or more suspected diagnoses, or while determining one or more suspected diagnoses, the remote computing resource(s) 118 may cause one or more actions to be performed. For instance, if the response(s) 214 include “I'd like to schedule an appoint with a Neurologist,” the remote computing resource(s) 118 may cause an indication to be stored in the user profile 126 of the patient. Additionally, or alternatively, if the question(s) ask “Does the patient have anxiety” and the response(s) indicates “Yes,” an indication may be stored in the user profile 126. This indication may cause doctors, clinics, and/or other specialists to contact the patient, may cause the remote computing resource(s) 118 to transmit a message to the patient to see a doctor, and so forth.

The user profile(s) 126 and/or the database(s) 128 may be updated based on the response(s) 214, such as symptoms indicated by the patient in the response(s) 214. Additionally, in some examples, the remote computing resource(s) 118 may obtain, retrieve, and/or receive the medical history 206, the personal information 208, and/or the third-party medical data 210 continuously from the third-party sources. In some examples, the remote computing resource(s) 118 may obtain, retrieve, and/or receive the medical history 206, the personal information 208, and/or the third-party medical data 210 at given time intervals. The given time intervals may include, but are not limited to, every minute, half-hour, hour, day, week, month, or the like.

Additionally, to protect the privacy of information contained in the user profile(s) 126, the remote computing resource(s) 118 may receive consent from patients to share, correlate, or otherwise use the information in determine one or more suspected diagnoses. That is, as noted above, the remote computing resource(s) 118 may correlate symptoms of one patient with symptoms or another patient in determining suspected diagnoses, question(s) 212 to ask the patient, or analyzing the response(s) 214. Before such correlation of comparisons, the remote computing resource(s) 118 may first receive consent.

### Illustrative Device

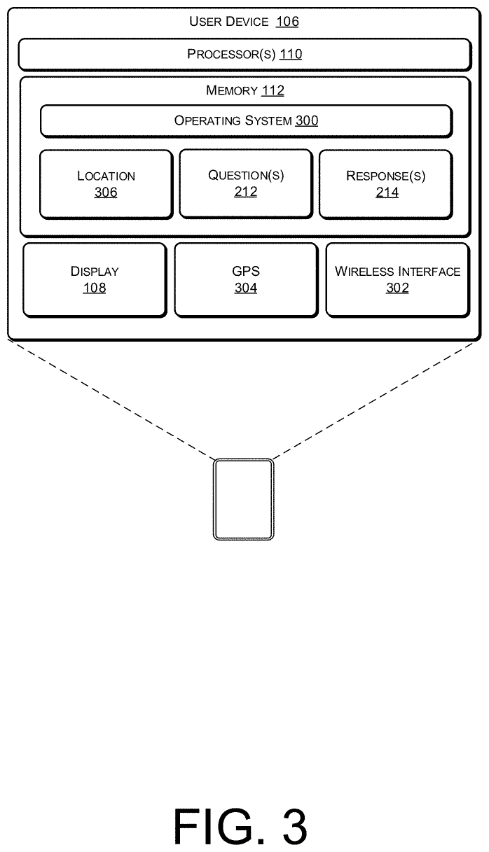

FIG. 3 shows selected functional components of the device 106. Generally, the device 106 may be implemented as a standalone device that is relatively simple in terms of functional capabilities with input/output components, memory (e.g., the memory 112), and processing capabilities. For instance, the device 106 may include the display 108 or a touchscreen to facilitate visual presentation (e.g., text, charts, graphs, images, etc.), graphical outputs, and receive user input through either touch inputs on the display 108 (e.g., virtual keyboard).

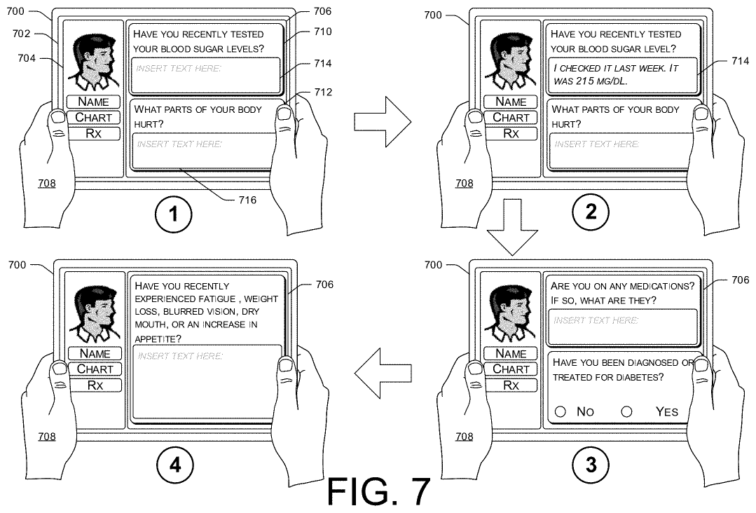

The memory 112 stores an operating system 300. The operating system 300 may configure the processor(s) 110 to display the question(s) 212 on the display 108. Display of the question(s) 212 may involve displaying text of the question(s) 212 and input fields where a user (e.g., provider 102) is able to respond to the question(s) 212, as shown and discussed below in FIG. 7. After typing, submitting, or otherwise entering a response 214 to the question(s) 212, the operating system 300 may generate and transmit the response(s) 214 to the remote computing resource(s) 118. In some instances, multiple questions 212 may be displayed in unison, or at the same time on the display 108, or only one question 212 may be presented at a time on the display 108. Further, the device 106 may be configured to transmit one or more response 214 at the same time, or response(s) 214 may be submitted individually.

In the illustrated example, the device 106 includes a wireless interface 302 to facilitate a wireless connection to a network (e.g., the network 120) and the remote computing resource(s) 118. The wireless interface 302 may implement one or more of various wireless technologies, such as WiFi, Bluetooth, RF, and the like.

FIG. 3 also illustrates that the device 106 may include global positioning systems (GPS) 304 or other locating devices may be used. The GPS 304 may generate a location 306 that corresponds to a location of the device 106. In some instances, the processor(s) 110 may utilize the location 306 in downloading or receiving the question(s) 212 from the remote computing resource(s) 118. For instance, the location 306 may indicate that the device 106 is within a residence of a patient or a threshold proximity thereof. In response, the processor(s) 110 may receive (e.g., download) the question(s) 212 from the remote computing resource(s) 118. In another instance, the location 306 may indicate the device 106 is traveling towards the residence of the patient, and in response, the device 106 may receive the question(s) 212. As noted above, however, to receive the question(s) 212, the processor(s) 110 may transmit a pull request, or the remote computing resource(s) 118 may push the question(s) 212 in response to determining the device 106 is within the residence or is in route to the patient's residence.

In some instances, the device 106 may include one or more microphones that receive audio input, such as voice input from the provider 102 and/or the patient 104, and one or more speakers to output audio. For instance, the provider 102 or the patient 104 may interact with the device 106 by speaking to it, and the one or more microphone captures the user speech. In response, the device 106 performs speech recognition (e.g., speech recognition engine and/or speech-to-text) and types text data into a field corresponding to the question(s) 212. Additionally, or alternatively, the audio data may be provided to the remote computing resource(s) 118 as the response(s) 214, where the remote computing resource(s) 118 analyzes the response(s) 214. To relay the question(s) 212 to the patient 104, the device 106 may emit audible statements through the speaker. In this manner, and in some instances, the provider 102 and/or the patient 104 may interact with the device 106 through speech, without using and/or in addition to the virtual keyboard presented on the display 108, for instance.

In some instances, the memory 112 may include the user profile(s) 126, the databases 128, the prediction analytics component 200, the question engine 202, and/or the feedback engine 204. Additionally, at least some of the processes of the remote computing resource(s) 118 may be executed by the device 106.

### Illustrative Processes

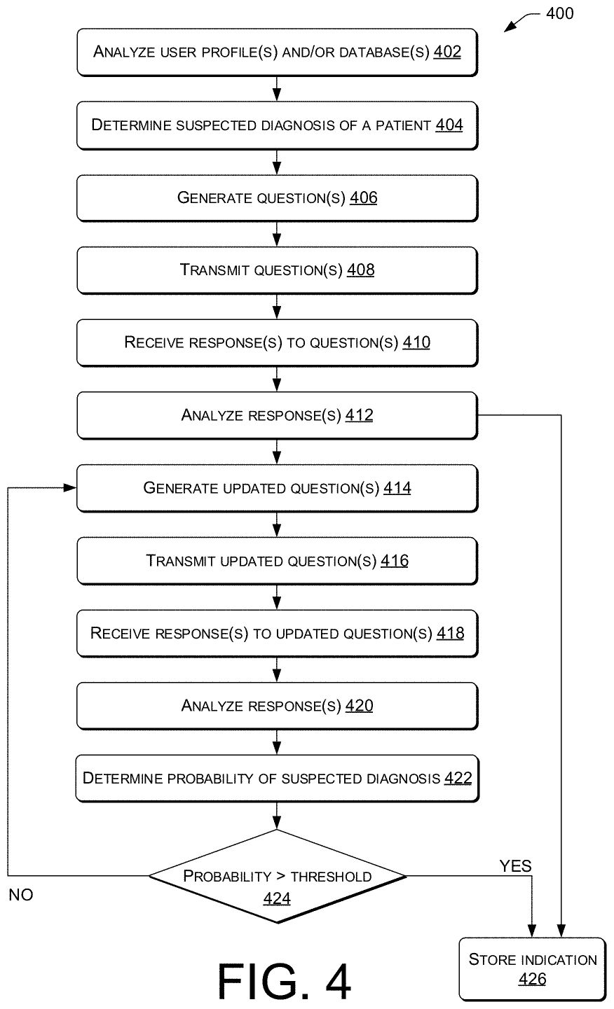

FIGS. 4-6 illustrate various processes related to dynamic prompting for diagnoses suspecting. The processes described herein are illustrated as collections of blocks in logical flow diagrams, which represent a sequence of operations, some or all of which may be implemented in hardware, software, or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process, or alternative processes, and not all of the blocks need be executed. For discussion purposes, the processes are described with reference to the environments, architectures and systems described in the examples herein, such as, for example those described with respect to FIGS. 1-3, 7, and 8, although the processes may be implemented in a wide variety of other environments, architectures and systems.

FIG. 4 illustrates a process 400 for determining suspected diagnoses through generating question(s) and receiving response(s). At block 402, the process 400 may analyze user profile(s) and/or database(s). For instance, the remote computing resource(s) 118 may analyze the user profile(s) 126 and/or the database(s) 128. The analysis at block 402 may be performed by the prediction analytics component 200 discussed hereinabove. In some instances, the block 402 may be performed in response to certain actions, such as a patient requesting an examination and/or a patient enrolling in a new health care plan.

At block 404, the process 400 may determine a suspected diagnosis, or diagnoses, of a patient. For instance, in analyzing the user profile(s) 126 and/or the database(s) 128 (e.g., the block 402), the remote computing resource(s) 118 may determine one or more suspected diagnoses of the patient. In some instances, the suspected diagnoses may include previous diagnoses of the patient (i.e., the patient continues to be diagnosed) or one or more diagnoses that the patient may or is likely to have (i.e., but has yet to be diagnosed). In this sense, in analyzing the user profile(s) 126 and/or the database(s) 128, the remote computing resource(s) 118 determines suspected diagnoses of the patient even though the patient may have yet to be diagnosed with an illness.

At block 406, the process 400 may generate question(s). For instance, the question engine 202 may generate question(s) 212 that relate to the one or more suspected diagnoses of the patient, relate to symptoms the patient is experiencing, or other health-related issues. Discussed above, the question(s) 212 are tailored to the one or more suspected diagnoses of the patient. For instance, with the question(s) 212, the process 400 is able to determine a probability of the patient having the one or more suspected diagnoses. That is, the question(s) 212 inquire about the probability associated with the patient having the one or more suspected diagnoses. Thus, through the question(s) 212, the process 400 determines an associated probability of the one or more suspected diagnoses. Additionally, or alternatively, the question(s) 212 may ask for a confirmation of the suspected diagnoses (e.g., “Does the patient have diabetes,” “Does the patient have anxiety,” etc.).

At block 408, the process 400 may transmit the questions. For instance, the remote computing resource(s) 118 may transmit the question(s) 212 to a device, such as the device 106. In response to receiving the question(s) 212, the device 106 is configured to display the question(s) 212. In some instances, the transmission may include a command that causes the device 106 to display the question(s) 212.

At block 410, the process 400 may receive response(s) to the question(s). For instance, after the question(s) 212 are answered, the device 106 may transmit the response(s) 214 to the remote computing resource(s) 118. In some instances, response(s) 214 to each question 212 may be received individually (e.g., one at a time) or response(s) 214 to one or more question(s) 212 may be received together (e.g., as a batch). The response(s) 214 may be received from the device 106, for instance, in response to a pull request by the remote computing resource(s) 118.

At block 412, the process 400 may analyze the response(s). Analysis may involve the feedback engine 204 decoding or deciphering the response(s) 214, for instance, using machine-learning techniques. Further, the analysis may involve determining a probability the patient has the one or more suspected diagnoses or confirming or denying the suspected diagnosis. That is, response(s) 214 to the question(s) 212 may be analyzed by the prediction analytics component 200 to determine a likelihood, probability, and/or whether the patient has the one or more suspected diagnoses. In some instances, this determination may involve comparing the response(s) 214 to the user profile(s) 126 and/or the database(s) 128. For instance, the response(s) 214 may indicate symptoms the patient is experiencing and these symptoms may be compared against the user profile(s) 126 and/or the database(s) 128 to determine additional suspected diagnoses and/or the probability the patient has the one or more suspected diagnoses, from the block 402. As a result, the remote computing resource(s) 118 may refine the questions, using the response(s) 214, to determine the one or more suspected diagnoses of the patient. Moreover, analysis of the response(s) 214 may include natural language understanding (NLU). For instance, the prediction analytics component 200 may analyze a response 214 including “I'm really sick” or “I'm feeling OK” and use these indications to determine additional information to inquire the patient about.

Further, if the response 214 to a question 212 asking “Does the patient have diabetes” is “No,” an indication may be stored in the profile of the patient. That is, from block 412, the process 400 may proceed to block 426 where an indication is stored in the profile of the patient (i.e., that the patient is not suspected of having diabetes). Noted above, in providing this response, the provider may use his or her judgement when examining the patient and asking questions related to determining whether the patient has diabetes (which may nor may not be generated by the question engine 202).

Additionally, or alternatively, in some instances, from block 412, the process may proceed to block 414, where the process 400 may generate updated question(s). For instance, using the results from the response(s) (e.g., the block 412), updated question(s) are generated by the question engine 202. The updated question(s) may take into consideration the response(s) 214 provided to further determine one or more suspected diagnoses of the patient.

At block 416, the process 400 may transmit the updated question(s). For instance, the remote computing resource(s) 118 may transmit the updated question(s) to the device 106, where the device 106 is configured to display the questions. At block 418, the process 400 may receive response(s) to the updated question(s). Thereafter, at block 420, the process 400 may analyze the response(s) to the updated question(s). In some instances, the analysis at the block 420 may involve a similar analysis as the block 412, such as comparing the response(s) 214 to the user profile(s) and/or the database(s).

The process 400 between the block 412, the block 414, and the block 416 may occur substantially close in time or contemporaneously with one another. More particularly, the analysis of the response(s) 214 and the transmittal of the updated question(s) allows the device 106 to receive and display updated questions based on the response(s) 214. In doing so, the updated questions are presented on the device 106 instead of or in lieu of continuing to display the initial questions (e.g., the questions generated at the block 406). In other words, the process 400 takes into consideration the response(s) 214 provided by the patient and generates the updated questions such that they are sent to the device before the device displays the initially generated questions (which may no longer be relevant based on analyzing the response(s). In some instances, the updated questions may be generated after each response, a series of responses, or other criteria. Additionally, the updated question(s) transmitted to the device 106 may replace all of the initial questions or only a certain portion thereof.

At block 422, the process 400 may determine a probability of the suspected diagnosis. For instance, as a result of the analysis of the response(s) 214, the process 400 may determine the probability that the patient has the suspected diagnosis. In some instances, the probability may be determined through comparing symptoms of the patient with the user profile(s) 126 and/or the databases 128. To illustrate, the response(s) 214 may indicate that the patient has normal blood sugar levels, a normal appetite, and is not experiencing blurred vision. With these response(s) 214, the remote computing resource(s) 118 may determine a probability of a suspected diagnosis (e.g., diabetes).

At block 424, the process 400 may determine whether the probability is greater than a threshold probability. In some instances, the threshold probability may represent more likely than not that the patient has a suspected diagnosis, beyond a reasonable doubt that the patient has the suspected diagnosis, by a preponderance of the evidence that the patient has the suspected diagnosis, or other standards. In some instances, the threshold probability may include a numerical value. For instance, the machine-learning techniques may determine a score associated with an illness, and specifically, a score as to whether the patient has the suspected diagnosis. This score may be compared to the threshold probability, which represents a threshold that a patient likely has the diagnosis. Accordingly, if the score, for instance, is above the threshold probability, it may be determined that the patient potentially has the suspected diagnosis.

If the determination at the block 424 does not meet the threshold probability, the process 400 may continue to the block 414, where updated question(s) are generated. That is, the process 400 between the block 414 and the block 424 may be repeated to analyze response(s), generate updated question(s), transmit the updated question(s), receive response(s) to the updated question(s), and determine whether the probability of a suspected diagnosis is greater than a threshold probability. In some instances, the updated question(s), from the block 424, may relate to the suspected diagnosis of the patient (e.g., the block 404) or may relate to other suspected diagnoses determined through analyzing the responses (e.g., the block 412 and/or the block 420). To illustrate, after analyzing the response(s) 214, the process 400 may determine to continue questing a patient about a first suspected diagnosis (e.g., diabetes) and/or may transition to questioning the patient about a second suspected diagnosis (e.g., epilepsy). For instance, after receiving and analyzing the response(s) 214, the process 400 may determine that the patient is not suspected to have diabetes (e.g., the block 424). Rather than continue generating question(s) 212 about diabetes, the remote computing resource(s) 118 may generate question(s) 212 that relate to determining whether the patient is suspected of having epilepsy. In some instances, the determination of generating questions relating to epilepsy may come by way of analyzing the responses (e.g., the responses may indicate symptoms and/or signs of epilepsy) or epilepsy may have been a suspected diagnosis of the patient (e.g., the block 404).

Comparatively, if at block 424 the process 400 determines that the probability is greater than the threshold, the process 400 may continue to the block 426. At the block 426, noted above, the remote computing resource(s) 118 may store an indication in the user profile 126 of the patient indicating the patient is suspected to have a diagnosis. In some instances, the indication may be used by medical professionals or other third-party sources such as contacting the patient.

Additionally, the indication stored at the block 426 may be used by the prediction analytics component 200 when determining suspected diagnoses of patients in the future.

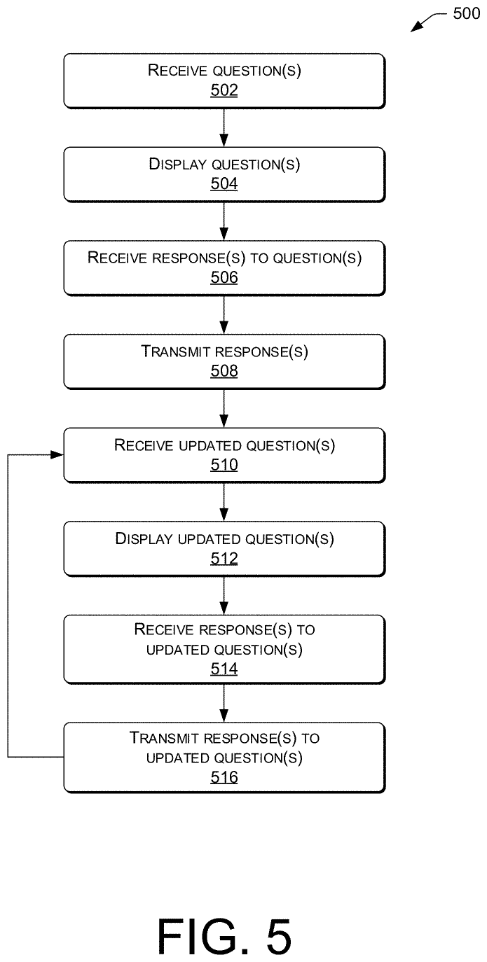

FIG. 5 illustrates a process 500 for receiving, transmitting, and displaying questions. At block 502, the process 500 may receive question(s). For instance, the device 106 may receive the question(s) 212 from the remote computing resource(s) 118. In some instances, the question(s) 212 may be received individually or as a group. Further, the question(s) 212 may be received in response to a pull command issued by the device 106. For instance, a location of the device 106 (e.g., the location 306) may indicate that the device 106 is within a residence of the patient, and in response, the question(s) 212 may be received from the remote computing resource(s) 118.

At block 504, the process 500 may display the question(s). For instance, the question(s) 212 may be displayed on the display 108 of the device 106. The device 106 may be configured to display any form and/or content relating to the question(s) 212, such as images, text, and so forth.

At block 506, the process 500 may receive response(s) to the question(s). For instance, the display 108 may include a GUI where a user (e.g., the provider 102) is able to enter the response(s) 214 to the question(s) 212 (e.g., text box, input field, etc.). For instance, the user may enter the response(s) 214 to the question(s) 212 using a virtual keyboard.

At block 508, the process 500 may transmit the response(s). For instance, the device 106 may transmit the response(s) 214 to the remote computing resource(s) 118 using the wireless interface 302. In some instances, the device 106 may transmit the response(s) 214 to the question(s) 212 individually, or the device 106 may transmit one or more of the response(s) 214 to the remote computing resource(s) 118 at the same time.

At block 510, the process 500 may receive updated question(s). For instance, the device 106 may receive updated question(s) from the remote computing resource(s) 118. Moreover, receipt of the updated question(s) may occur close in time or substantially contemporaneously with transmitting the response(s) 214, such that the device 106 receives, and displays, the updated question(s) before continuing to display the initial question(s).

At block 512, the process 500 may display the updated question(s). For instance, the device 106 may display the updated question(s) on the display 108.

At block 514, the process 500 may receive response(s) to the updated question(s). For instance, the device 106 may receive response(s) to the updated question(s) through the user typing into an input field.

At block 516, the process 500 may transmit the response(s) to the updated question(s). For instance, the device 106 may transmit the response(s) to the remote computing resource(s) 118.

From block 516, the process 500 may continue to the block 510 where the process 500 may receive updated question(s). As such, the device 106 may continuously receive updated question(s) from the remote computing resource(s) 118, display the updated question(s), receive response(s) to the updated question(s), and transmit the response(s) to the updated question(s). The loop shown by the arrowed-line from the block 516 and the block 510 may be repeated for any number of instances.

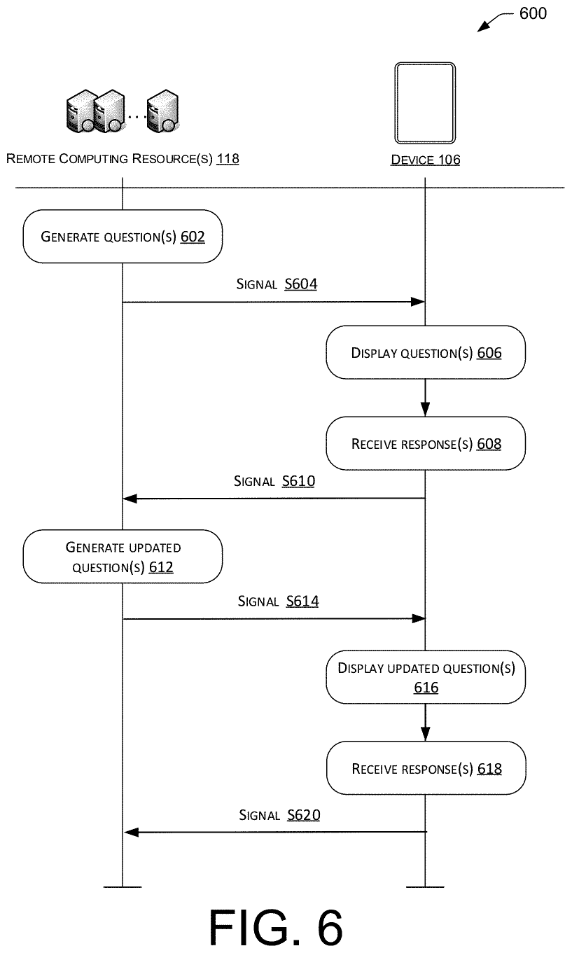

FIG. 6 illustrates a signal diagram of a process 600 for generating question(s) using remote computing resource(s) 118 and displaying the question(s) and receiving response(s) using a device 106.

At block 602, the process 600 may generate question(s). In some instances, the block 602 may be similar to and/or represent the block 406 of the process 400. For instance, the remote computing resource(s) 118 may generate the question(s) 212 after analyzing the user profile(s) 126 and the database(s) 128.

At signal S604, the process 600 may transmit a signal. For instance, the remote computing resource(s) 118 may transmit the signal S604 to the device 106. In some instance, the signal S604 may include the question(s) 212 as well as a command that causes the device 106 to display the question(s) 212. In some instances, the signal S604 may be similar to and/or represent the block 408 of the process 400.

At block 606, the process 600 may display the question(s). For instance, the device 106 may display the question(s) 212 as included within the signal S604. In some instances, in response to receiving the signal S604, the device 106 displays the question(s) 212. That is, the signal S604 may cause the device 106 to display the question(s) 212. Further, in some instances, the block 606 may be similar to and/or represent the block 504 of the process 500.

At block 608, the process 600 may receive response(s) to the question(s). For instance, after displaying the question(s) 212, the device 106 may receive the response(s) 214. In some instances, the block 608 may be similar to and/or represent the block 506 of the process 500.

At signal S610, the process 600 may transmit a signal. For instance, the device 106 may transmit the signal S610 to the remote computing resource(s) 118. In some instances, the signal S610 may include the response(s) 214 as well as a command that causes the remote computing resource(s) 118 to analyze the response(s) 214 (e.g., the block 412 of the process 400). In some instances, the signal S610 may be similar to and/or represent the block 508 of the process 500.

At block 612, the process 600 may generate updated question(s). For instance, the remote computing resource(s) 118 may receive the response(s) 214 as included within the signal S610 and generate updated question(s). In some instances, the signal S610 may cause the remote computing resource(s) 118 to analyze the response(s) 214 (e.g., the block 412 of the process 400) to determine content of the updated question(s). Further, in some instances, the block 612 may be similar to and/or represent the block 414 of the process 400.

At signal S614, the process 600 may transmit a signal. For instance, the remote computing resource(s) 118 may transmit the signal S614 to the device 106. In some instances, the signal S614 may include the updated question(s) as well as a command that causes the device 106 to display the updated question(s). In some instances, the signal S614 may be similar to and/or represent the block 416 of the process 400.

At block 616, the process 600 may display the updated question(s). For instance, the device 106 may display the updated question(s) as included within the signal S614. In some instances, in response to receiving the signal S614, the device 106 displays the updated question(s). The block 616 may be similar to and/or represent the block 512 of the process 500.

At block 618, the process 600 may receive response(s) to the updated question(s). For instance, after displaying the updated question(s), the device 106 may receive response(s) to the updated question(s). In some instances, the block 618 may be similar to and/or represent the block 514 of the process 500.

At signal S620, the process 600 may transmit a signal. For instance, the device 106 may transmit the signal S620 to the remote computing resource(s) 118. In some instances, the signal S620 may include the response(s) to the updated question(s) as well as a command that causes the remote computing resource(s) 118 to analyze the response(s). In some instances, the signal S620 may be similar to and/or represent the block 516 of the process 500.

After transmitting the signal 620, the process 600 may continue to further generate updated question(s) and receive responses to the updated question(s), as discussed herein above in the process 400 and/or the process 500.

FIG. 7 illustrates an iterative process of displaying questions and receiving responses on a device 700 (which may be similar to and/or represent the device 106). The progression of the process shown in FIG. 7 is illustrated by the arrows.

The device 700 is shown including a display 702 having a first area 704 and a second area 706. In the first area 704, background information of a patient is displayed. For instance, the first area 704 may include an image of the patient, a name of the patient, medical charts of the patient, or prescriptions of the patient. However, while FIG. 7 illustrates certain background information, other information may be displayed as well, or the background information may be presented differently than shown. The background information may be accessed through a user 708 interacting with the display 702. For instance, the user 708 may select “Chart” within the first area and medical charts of the patient may be displayed on the display 702.

Shown at “1,” the second area 706 displays a first question within a first field 710 and a second question within a second field 712. As noted above, the first question and the second question may be specific to the patient. For instance, as a result of analyzing the user profile(s) and/or the database(s), the patient may be suspected of having diabetes, and accordingly, the first question asks the patient “Have you recently tested your blood sugar levels?” The second question asks the patient “What parts of your body hurt?” which may or may not relate to diabetes but may instead relate to another suspected diagnosis of the patient or a general health inquiry of the patient.

The user 708 may respond to the first question using a first input field 714 and may respond to the second question using a second input field 716. Through touching an area within the first input field 714 or the second input field 716, respectively, the user 708 may type a response (e.g., using a virtual keyboard that pops up). That is, at “2” the user 708 has entered, in response to the first question, “I checked it last week. It was 215 mg/dL.” After entering this response, the device 700 may transmit, whether automatically or upon a selection by the user 708, the response (e.g., the response(s) 214) to the remote computing resource(s) 118.

As shown at “3”, the device 700 displays updated question(s) in the second area 706. The updated question(s) may be generated based at least in part on the response at “2”. That is, the response of “215 mg/dL” may be analyzed to determine that the patient has a high blood sugar level, and hence, updated question(s) may be asked of the user inquiring about diabetes. More particularly, the updated question(s) ask the patient a third question “Are you on any medications? If so, what are they?” and a fourth question “Have you been diagnosed or treated for diabetes?” The updated question(s) therefore identify, through continued response(s) of the user 708, whether diabetes, for instance, is a suspected diagnosis. As a result, using the responses, a probability that the patient has diabetes, for instance, may be determined.

Shown between “2” and “3”, the user 708 does not respond to the second question (“What parts of your body hurt?”). Instead, before permitting the user 708 to respond to the second question, the second question may be updated with updated question(s), as the updated questions may relate more appropriately to determining suspecting diagnoses of the patient (e.g., “Have you been diagnosed or treated for diabetes?”). That is, rather than continuing to display the second question, which may no longer be appropriate with respect to the suspected diagnoses given the response to the first question, updated question(s) may instead be asked of the patient. In other words, the response(s) entered may be used to continuously update question(s) presented on the device 700.

Continuing with this example, after responding to the third questions and the fourth question, or at least one of the third question or the fourth question, a fifth question at “4” is presented in the second area 706. Note that between “3” and “4”, the process of the user 708 entering a response is not shown. However, entering a response to the third question and the fourth question may be similar to that illustrated and discussed between “2” and “3”. The fifth question further relates to diabetes, indicating that the patient may be suspected of having diabetes.

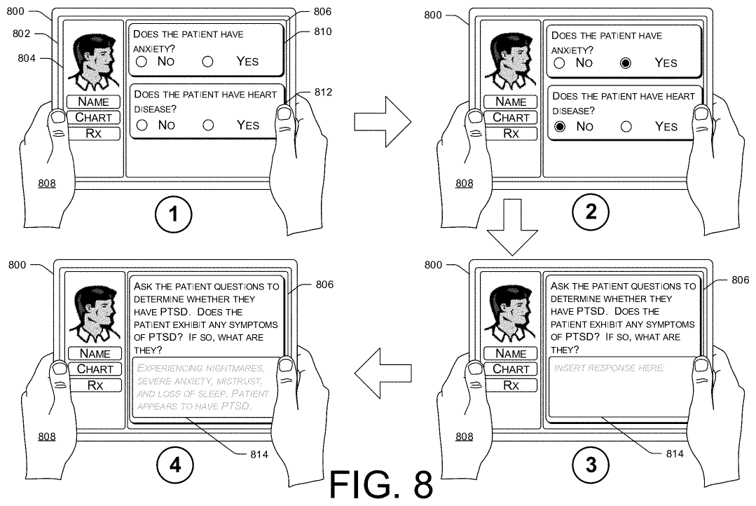

FIG. 8 illustrates an iterative process of displaying suspected diagnoses and receiving responses on a device 800 (which may be similar to and/or represent the device 106 and/or the device 700). The progression of the process shown in FIG. 8 is illustrated by the arrows. The device 800 may include a display 802 having a first area 804 which displays background information of a patient and a second area 806. A user 808 may interact with the device 800 and the display 802.

Shown at “1,” the second area 804 displays a first question within a first field 810 and a second question within a second field 812. As noted above, the first question and the second question may be specific to the patient as a result of analyzing the user profile(s) 126 and/or the database(s) 128. That is, as a result of the analysis, the patient may be suspected of having anxiety and heart disease, and accordingly, the first question asks the user “Does the patient have anxiety?” while the second question asks “Does the patient have heart disease?” As a result of being prompted to answer these questions, the user 808 (e.g., the provider 102) may examine the patient to determine whether patient has anxiety and/or heart disease. For instance, the user 808 may perform tests on the patient and/or may ask questions of the patient. In some instances, the questions may be generated by the question engine 202, as discussed hereinabove.

At “2,” the user 808 may respond to the first question and may respond to the second question. Through touching an area within display, respectively, the user 808 may enter a response of “No” or “Yes.” That is, at “2” the user 808 has entered, in response to the first question, “Yes” and “No” in response to the second question. In entering these responses, the user 808 may rely on his or her medical training, education, and his or her examination of the patient. That is, after examining or assessing the patient, the user 808 determines (or makes a judgment) that the patient has anxiety but does not have heart disease. As a result, the device 800 may transmit these responses to the remote computing resource(s) 118, whereby indications are stored in the profile of the patient. The indications may further be used for future diagnoses suspecting.

In some instances, from “2,” the device 800 may display additional suspected diagnoses of the patient. For instance, at “3” the second area 806 prompts the user 808 to inquire about PTSD. Here, the device 800 displays in the second area 806, an input field 814 that allows the user 808 may respond to the inquiry.

At “4,” the user 808 enters a response in the input field 814. After entering the response, the device may transmit the response to the remote computing resource(s) 118, where the prediction analytics component 200 analyzes the response. For instance, with the given response shown at “4,” the prediction analytics component 200 may determine a suspected diagnosis (e.g., PTSD). In some instances, this determination may rely on the user's 808 judgment and assessment.

### CONCLUSION

While the foregoing invention is described with respect to the specific examples, it is to be understood that the scope of the invention is not limited to these specific examples. Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Although the application describes embodiments having specific structural features and/or methodological acts, it is to be understood that the claims are not necessarily limited to the specific features or acts described. Rather, the specific features and acts are merely illustrative some embodiments that fall within the scope of the claims of the application.

What is claimed is:

1. A system comprising:
one or more processors; and
computer-readable media storing first computer-executable instructions that, when executed by the one or more processors, cause the one or more processors to perform operations comprising:
analyzing, using one or more machine learning techniques, a user profile of a patient, the user profile including at least a medical history of the patient;
determining, based at least in part on analyzing the user profile of the patient, a first suspected diagnosis of the patient and a second suspected diagnosis of the patient;
generating, based at least in part on determining the first suspected diagnosis, first data corresponding to a first question, wherein the first question is related to the first suspected diagnosis of the patient;
generating, based at least in part on determining the second suspected diagnosis, second data corresponding to a second question, wherein the second question is related to the second suspected diagnosis of the patient;
transmitting the first data and the second data to a remote device associated with a medical professional engaged in examining the patient, wherein the remote device is configured to display the first question and the second question;
receiving, from the remote device, first input data representing a first response to the first question and second input data representing a second response to the second question;
analyzing the first response to the first question and the second response to the second question;
determining, based at least in part on analyzing the first response and the second response, that the patient is diagnosed with the first suspected diagnosis; and
storing an indication in the user profile of the patient, wherein the indication indicates the patient is diagnosed with the first suspected diagnosis.

2. The system of claim 1, wherein the indication is a first indication, and wherein the one or more non-transitory computer-readable media store second computer-executable instructions that, when executed by the one or more processors, cause the one or more processors to perform operations comprising:
determining, based at least in part on analyzing the first response and the second response, that the patient is not diagnosed with the second suspected diagnosis; and
storing a second indication in the user profile of the patient, wherein the second indication indicates the patient is not diagnosed with the second suspected diagnosis.

3. The system of claim 1, wherein the one or more machine learning techniques:
determine a first correlation between a first indication of a first event stored in the user profile and the first suspected diagnosis; and
determining a second correlation between a second indication of a second event stored in the user profile and the first suspected diagnosis.

4. The system of claim 3, wherein determining the first suspected diagnosis is based at least in part on the first correlation and the second correlation being above a threshold probability level, and wherein individually:
the first correlation is not above the threshold probability level; and
the second correlation is not above the threshold probability level.

5. A method comprising:
analyzing, using one or more suspected diagnoses models, one or more user profiles, wherein individual user profiles of the one or more user profiles correspond to a patient;
determining, based at least in part on analyzing the one or more user profiles, a first suspected diagnosis of a first patient;
generating, based at least in part on determining the first suspected diagnosis of the first patient, data corresponding to a question, wherein the question is related to either confirming or denying the first suspected diagnosis;
transmitting the first data to a remote device configured to display the question;
receiving, from the remote device, input data representing a response to the question;
analyzing the input data and the one or more user profiles;
generating, based at least in part on analyzing the input data and the one or more user profiles, one or more updated suspected diagnoses models, wherein the one or more updated suspected diagnoses models is used to determine a second suspected diagnosis of a second patient.

6. The method of claim 5, wherein the data is first data, the question is a first question, and the input data is first input data, the method further comprising:
generating, based at least in part on receiving the first input data, second data corresponding to a second question, wherein the second question is related to the first suspected diagnosis;
transmitting, to the remote device, the second data, wherein the remote device is configured to display the second question;
receiving, from the remote device, second input data representing a response to the second question; and
analyzing the second input data and the one or more user profiles, and
wherein generating the one or more updated suspected diagnoses models is based at least in part on analyzing the second input data.

7. The method of claim 5, further comprising determining, based at least in part on receiving the input data, that the patient is diagnosed with the first suspected diagnosis.

8. The method of claim 5, further comprising:
determining that a confidence level associated with the first suspected diagnosis is greater than a threshold confidence level, and
wherein determining the first suspected diagnosis of the first patient is based at least in part on the confidence level being greater than the threshold confidence level.

9. The method of claim 5, further comprising, based at least in part on analyzing the input data, storing an indication of the first suspected diagnosis in a user profile corresponding to the first patient, wherein the indication is used to at least one of:
recommend a doctor to visit the first patient;
recommend a visitation schedule for the first patient; or
diagnose the first patient.

10. The method of claim 5, further comprising:
determining a first correlation between one or more first characteristics of the patient and the first suspected diagnosis; and
determining a second correlation between one or more second characteristics of the patient and the first suspected diagnosis, and
wherein determining the first suspected diagnosis of the patient is based at least in part on the first correlation and the second correlation.

11. The method of claim 10, wherein:
determining the first suspected diagnosis is based at least in part on a confidence associated with the first suspected diagnosis being greater than a first confidence level;
the first correlation is associated with a second confidence level, the second confidence level being less than the first confidence level;
the second correlation is associated with a third confidence threshold level, the third confidence level being less than the first confidence level; and
collectively, the first correlation and the second correlation are above the first confidence level.

12. The method of claim 5, further comprising:
determining, based at least in part on analyzing the one or more user profiles, a first probability of the first suspected diagnosis of the first patient;
determining, based at least in part on analyzing the one or more user profiles, a second probability of a third suspected diagnosis of a first patient; and
determining that the first probability is greater than the second probability, and
wherein generating the data corresponding to the question is based at least in part on the first probability being greater than the second probability.

13. The method of claim 5, further comprising transmitting, based at least in part on receiving the input data, a request to contact the first patient.

14. A system comprising:
at least one processor; and
one or more non-transitory computer-readable media storing first computer-executable instructions that, when executed by the at least one processor, cause the at least one processor to perform acts comprising:
analyzing, using one or more machine learning techniques, one or more databases, wherein the one or more databases include a profile associated with a patient;
generating, based at least in part on analyzing the one or more databases, questions;
transmitting the questions to a remote device, wherein the remote device is configured to display one or more of the questions;
receiving, from the remote device, first feedback to a question of the questions;
generating, based at least in part on the first feedback, updated questions; and
transmitting the updated questions to the remote device, wherein the remote device is configured to display one or more of the updated questions.

15. The system of claim 14, wherein transmitting the updated questions is performed substantially contemporaneously with receiving the first feedback.

16. The system of claim 14, wherein the updated questions comprises first updated questions, and wherein the one or more non-transitory computer-readable media store second computer-executable instructions that, when executed by the one or more processors, cause the one or more processors to perform operations comprising:
receiving, from the remote device, second feedback to a question of the updated questions;
generating, based at least in part on the second feedback, second updated questions; and
transmitting the second updated questions to the remote device, wherein the remote device is configured to display one or more of the second updated questions.

17. The system of claim 16, wherein the one or more non-transitory computer-readable media store second computer-executable instructions that, when executed by the one or more processors, cause the one or more processors to perform operations comprising:
receiving, from the remote device, second feedback to a question of the first updated questions; and
determining, based at least in part on the second feedback, a first potential diagnosis of a patient
receiving, from the remote device, third feedback to a question of the second updated questions; and
determining, based at least in part on the third feedback, a second potential diagnosis of the patient.

18. The system of claim 14, wherein analyzing the one or more databases comprises determining a first event associated with the patient and a second event associated with the patient, wherein the first event and the second event fail to indicate a potential diagnosis of the patient, and wherein the one or more non-transitory computer-readable media store second computer-executable instructions that, when executed by the one or more processors, cause the one or more processors to perform operations comprising:
weighting, using one or more machine learning techniques, the first event, the second event, the first input data, and the second input data, and
wherein determining the diagnosis of the patient is based at least in part on weighting the first event, the second event, the first input data, and the second input data.

19. The system of claim 14, wherein the one or more non-transitory computer-readable media store second computer-executable instructions that, when executed by the one or more processors, cause the one or more processors to perform operations comprising:
analyzing the first feedback;
determining, based at least in part on analyzing the first feedback, a first probability of a potential diagnosis;
receiving, from the remote device, second feedback to a question of the updated questions;
analyzing the second feedback;
determining, based at least in part on analyzing the second feedback, a second probability of the potential diagnosis;
determining that the second probability is greater than the first probability; and
storing, based at least in part on the second probability being greater than the first probability, an indication of the potential diagnosis in the profile of the patient.

20. The system of claim 19, wherein the one or more non-transitory computer-readable media store second computer-executable instructions that, when executed by the one or more processors, cause the one or more processors to perform operations comprising:
determining, based at least in part on analyzing the one or more databases, one or more potential diagnosis of the patient, and
wherein the potential diagnosis of the patient is one of the one or more potential diagnosis of the patient.
